(19) United States
(12) Patent Application Publication
(10) Pub. No.: US 2023/0094014 A1
(43) Pub. Date: Mar. 30, 2023

(54) COMPOSITIONS COMPRISING HYDROLYSED PROTEINS

(71) Applicants: Croda International Plc, Goole, East Yorkshire (GB); Croda, Inc., Plainsboro, NJ (US)

(72) Inventors: Neil James, Goole, East Yorkshire (GB); Kimun Park, Plainsboro, NJ (US)

(73) Assignees: Croda International Plc, Goole, East Yorkshire (GB); Croda, Inc., Plainsboro, NJ (US)

(21) Appl. No.: 17/801,034

(22) PCT Filed: Feb. 26, 2021
(86) PCT No.: PCT/US2021/019848

§ 371 Date: Aug. 19, 2022

Related U.S. Application Data
(60) Provisional application No. 62/982,862, filed on Feb. 28, 2020.

(51) Int. Cl. A61K 8/64; A61Q 5/00

(52) U.S. Cl. CPC A61K 8/645; A61Q 5/002; A61K 2800/5922

(57) ABSTRACT

The present invention provides a composition comprising a blend of: a) a first component which is partially hydrolysed protein obtained from a potato protein source; and b) a second component which is partially hydrolysed protein obtained from a non-potato vegetable protein source; wherein the weight ratio of first component:second component in the blend is in the range 0.5:1 to 2:1. The invention also provides a personal care formulation comprising such a composition and a personal care ingredient, wherein the personal care formulation is for topical application to skin or hair. The invention further provides a method of treating hair using a composition and the use of a composition for the treatment of hair.

### CROSS-REFERENCE TO RELATED APPLICATION

This application claims priority to U.S. Provisional Application No. 62/982,862, titled “COMPOSITIONS COMPRISING HYDROLYSED PROTEINS”, filed Feb. 28, 2020, and the contents of which are incorporated herein by reference in their entireties for all purposes.

### FIELD OF THE INVENTION

The present invention relates to compositions comprising hydrolysed proteins derived from vegetable sources and their use in personal care formulations, in particular for application to human skin or hair, preferably in hair care formulations. More particularly, the invention relates to a composition comprising a first hydrolysed protein component obtained from a potato source and a second hydrolysed protein component obtained from a non-potato vegetable source.

### BACKGROUND

The need for hair care products is well-known. Such products may be used for cleaning or styling human hair or addressing various problems encountered with human hair, for example, by treatments such as bleaching or colouring or by environmental factors, such as sun, heat and pollution. Some hair care products are also directed at improving qualities of the hair such as strength, shine or ease of brushing/combing.

Hydrolysed keratin protein is known as an ingredient for use in hair care formulations to strengthen hair and/or reduce breakage. It is generally accepted that the cystine/cysteine amino acid fraction of keratin protein makes a significant contribution to its strengthening properties. A distinguishing feature of keratin protein is the presence of large amounts of the sulfur-containing amino acid cysteine, required for the disulfide bridges that confer additional strength and rigidity to the protein by crosslinking. Human hair is approximately 14 wt % cysteine. However, hydrolysed keratin protein is derived from an animal source which can be undesirable for consumers.

There is a continual need for the development of improved personal care products and formulations, in particular hair care formulations, and ingredients.

### SUMMARY OF THE INVENTION

It is an object of the present invention to address the above and/or other disadvantages associated with the prior art.

According to a first aspect, the present invention provides a composition comprising a blend of:
a) a first component which is partially hydrolysed protein obtained from a potato protein source; and
b) a second component which is partially hydrolysed protein obtained from a non-potato vegetable protein source;
wherein the weight ratio of first component:second component in the blend is in the range 0.5:1 to 2:1.

According to a second aspect, the present invention provides a method of treating hair comprising the steps of:
i) applying to hair a composition comprising a first partially hydrolysed protein component obtained from a potato protein source and a second partially hydrolysed protein component obtained from a non-potato vegetable protein source; and
ii) optionally, rinsing the composition off the hair.

The composition of the second aspect may comprise any of the features of the first aspect of the invention.

According to a third aspect, the present invention provides the use of a composition comprising a first hydrolysed protein component obtained from a potato protein source and a second hydrolysed protein component obtained from a non-potato vegetable protein source for the treatment of hair.

The composition of the third aspect may comprise any of the features of the first aspect of the invention.

All of the features described herein may be combined with any of the above aspects, in any combination.

The present invention is based in part on the recognition by the inventors that the composition of the invention has advantageous properties due to its particular combination of potato derived protein and vegetable (non-potato) derived protein components. One advantageous property of this combination is a strengthening effect on human hair, for example an increase in the total work to break hair. Without being bound by theory, it is proposed that the particular combination and/or proportion of the first and second protein components provides an amino acid profile which contributes to the strengthening effect on human hair. Surprisingly, this strengthening effect is achieved despite the combination of the hydrolysed potato protein and the hydrolysed non-potato vegetable protein having a lower amount of cystine/cysteine amino acid than is normally present in a hydrolysed keratin protein.

### DETAILED DESCRIPTION OF THE INVENTION

It will be understood that any upper or lower quantity or range limit used herein may be independently combined.

It will be understood that, when describing the number of carbon atoms in a substituent group (e.g. ‘C1 to C6’), the number refers to the total number of carbon atoms present in the substituent group, including any present in any branched groups. Additionally, when describing the number of carbon atoms in, for example fatty acids, this refers to the total number of carbon atoms including the one at the carboxylic acid, and any present in any branch groups.

The term ‘personal care’ means human personal care. The term ‘hair care’ means human hair care.

The term ‘partially hydrolysed protein’ is used herein to mean proteins which have been subject to partial hydrolysis. The partially hydrolysed protein may comprise protein fragments, polypeptides, peptides, amino acids and/or peptones. It is to be understood that partial hydrolysis excludes hydrolysing the protein completely to consist solely of individual amino acids. The partially hydrolysed proteins may be produced by acid, alkali, and/or enzyme hydrolysis of native proteins. Enzyme hydrolysis is preferred. Without being bound by theory, an advantage of enzyme hydrolysis when compared with acid or alkali hydrolysis may be that the enzyme hydrolysis is more selective in the sites on the protein which are hydrolysed, thus producing a different set of protein fragments, polypeptides, peptides, amino acids and/or peptones than acid or alkali hydrolysis. In general, acid hydrolysis may produce the smallest fragments, alkali hydrolysis may produce the largest fragments, while enzyme hydrolysis may produce fragments of intermediate size between acid and alkali hydrolysis.

The or each hydrolysed protein may independently be further chemically modified, for example where the protein has been covalently reacted with a functional group such as a silane, a quaternary ammonium compound and/or an acid chloride.

One or more of the hydrolysed proteins may be a chemically unmodified hydrolysed protein. The term ‘chemically unmodified hydrolysed protein’ means a protein that has not been further chemically modified (or reacted) other than by hydrolysis. The first component may be a chemically unmodified hydrolysed protein. The second component may be a chemically unmodified hydrolysed protein. Preferably the first component and/or second component is a chemically unmodified hydrolysed protein.

It will be understood that the amino acid profile of the blend of the invention may be readily determined by hydrolysing the blend completely to individual amino acids then quantifying the amount of each amino acid present. The amino acid profile of the protein would be understood to remain the same or substantially the same from protein starting material to hydrolysed protein.

Many of the chemicals which may be used to produce the composition of the present invention are obtained from natural sources. Such chemicals typically include a mixture of chemical species due to their natural origin. Due to the presence of such mixtures, various parameters defined herein can be an average value and may be non-integral.

Preferably, the composition of the invention is substantially free from quaternary ammonium compounds. By the use of the term “substantially free from”, it is meant that the composition comprises preferably less than 10% by weight, more preferably less than 5% by weight, yet more preferably less than 2% by weight and most preferably, less than 1% by weight based on the total weight of the composition. Preferably, the composition comprises no quaternary ammonium compounds.

Preferably, the composition of the invention is substantially free from silicone compounds. By the use of the term “substantially free from”, it is meant that the composition comprises preferably less than 10% by weight, more preferably less than 5% by weight, yet more preferably less than 2% by weight and most preferably, less than 1% by weight based on the total weight of the composition. Preferably, the composition comprises no silicone compounds.

The composition of the invention may be a topical composition, preferably a topical composition for application to skin or hair (i.e. human skin or hair), more preferably a topical composition for application to hair. The hair may be natural, bleached, straightened or otherwise chemically treated. The hair may be African, Asian, European, Caucasian, Hispanic or another hair type. The hair may be naturally straight, wavy, curly or coily. The composition of the invention may be a cosmetic composition. The composition may not be a food composition. The composition may not be a (human) food product.

The composition of the invention comprises a blend of:
a) a first component which is partially hydrolysed protein obtained from a potato source; and
b) a second component which is partially hydrolysed protein obtained from a non-potato vegetable source;
wherein the weight ratio of first component:second component in the blend is in the range 0.5:1 to 2:1.

Preferably the composition does not comprise a protein component obtained from an animal protein source. This is advantageous since animal sources can be undesirable for consumers. Preferably the composition comprises no animal derived components. Preferably the composition is suitable for vegan consumers.

The first component is partially hydrolysed protein obtained from a potato source. The potato protein source may be a potato protein concentrate and/or isolate. An aqueous dispersion of the potato protein concentrate and/or isolate may be made as a first step and the protein may be hydrolysed as a second step. A difference between the potato protein source and the partially hydrolysed protein may be that the partially hydrolysed protein is more soluble in water at a reference temperature (e.g. room temperature) than the potato protein source.

The first component may be produced by acid, alkali or enzyme hydrolysis. Enzyme hydrolysis is preferred. One or more enzymes may be used. The enzyme is preferably not animal derived. Preferably the enzyme is from a micro-organism source. The enzyme(s) may comprise a carbohydrase and/or a protease. The hydrolysis may be performed to the extent required to achieve the desired weight average molecular weight of the hydrolysed protein. The extent of hydrolysis may be varied by varying the temperature, acid/alkali/enzyme used, and time taken. The resulting hydrolysed protein may be filtered and/or treated to remove undesired material. For example the hydrolysed protein may be treated to remove any chloride ions present if acid hydrolysis is used.

The weight average molecular weight (Mw) of the hydrolysed protein of the first component may be at least 200 Daltons (Da), preferably at least 300 Da, more preferably at least 400 Da, particularly at least 500 Da. The weight average molecular weight may be at most 100,000 Da, preferably at most 50,000 Da, more preferably at most 10,000 Da, particularly at most 5,000 Da, especially at most 2,500 Da. The molecular weight may be measured by size exclusion chromatography such as size-exclusion HPLC (SE-HPLC) as described in the Test Methods below.

The second component is partially hydrolysed protein obtained from a non-potato vegetable source. The non-potato vegetable source may be selected from wheat, cottonseed, pea and soya, preferably selected from wheat, cottonseed and pea, more preferably selected from cottonseed and pea, particularly selected from pea. Preferably the second component is obtained from a pea or cottonseed protein source. Preferably the composition does not comprise a protein component obtained from a wheat protein source. This may be advantageous since wheat protein may be undesirable for certain consumers.

The second component may be obtained from a protein concentrate and/or isolate. An aqueous dispersion of the protein concentrate and/or isolate may be made as a first step and the protein may be hydrolysed as a second step. A difference between the second protein source and the partially hydrolysed protein may be that the partially hydrolysed protein is more soluble in water at a reference temperature (e.g. room temperature) than the second protein source.

The second component may be produced by acid, alkali or enzyme hydrolysis. Enzyme hydrolysis is preferred. One or more enzymes may be used. The enzyme is preferably not animal derived. Preferably the enzyme is from a micro-organism source. The enzyme(s) may comprise a carbohydrase and/or a protease, preferably the enzyme is a protease. The hydrolysis may be performed to the extent required to achieve the desired weight average molecular weight of the hydrolysed protein. The extent of hydrolysis may be varied by varying the temperature, acid/alkali/enzyme used, and time taken. The resulting hydrolysed protein may be filtered and/or treated to remove undesired material. For example the hydrolysed protein may be treated to remove any chloride ions present if acid hydrolysis is used.

The weight average molecular weight (Mw) of the hydrolysed protein of the second component may be at least 400 Daltons (Da), preferably at least 500 Da, more preferably at least 600 Da, particularly at least 700 Da. The weight average molecular weight may be at most 100,000 Da, preferably at most 50,000 Da, more preferably at most 10,000 Da, particularly at most 5,000 Da, especially at most 2,500 Da. The molecular weight may be measured by size exclusion chromatography such as size-exclusion HPLC (SE-HPLC) as described in the Test Methods below.

The weight ratio of first component:second component in the blend (or in the composition) is at least 0.5:1, preferably at least 0.6:1, more preferably at least 0.7:1, particularly at least 0.8:1. The weight ratio of first component:second component in the blend is at most 2:1, preferably at most 1.8:1, more preferably at most 1.6:1, particularly at most 1.4:1, desirably at most 1.2:1. Preferably the weight ratio of first component to second component in the blend is in the range 0.6:1 to 1.4:1.

The blend may have a component amino acid profile which is characterised by the amount of individual amino acids present in a completely hydrolysed sample of the blend. The amino acid profile may be determined by completely hydrolysing the blend and quantifying the amount of each individual amino acid by high performance liquid chromatography (HPLC). The blend may comprise at least 5%, preferably at least 6%, more preferably at least 7% and/or preferably at most 20%, more preferably at most 16% aspartic acid by weight on the basis of the total weight of amino acids in the blend. The blend may comprise at least 10%, preferably at least 12%, more preferably at least 14% and/or preferably at most 28%, more preferably at most 26% glutamic acid by weight on the basis of the total weight of amino acids in the blend. The blend may comprise at least 5%, preferably at least 6%, more preferably at least 7% and/or preferably at most 15%, more preferably at most 10% leucine by weight on the basis of the total weight of amino acids in the blend. The blend may comprise at least 3%, preferably at least 4%, more preferably at least 5% and/or preferably at most 12%, more preferably at most 10% arginine by weight on the basis of the total weight of amino acids in the blend.

A surprising aspect of the invention is the benefits provided by the hydrolysed protein blend despite a low level of cystine/cysteine in the blend. The blend may comprise at most 10%, preferably at most 8%, more preferably at most 6%, particularly at most 4% cystine/cysteine by weight on the basis of the total weight of amino acids in the blend.

The weight average molecular weight (Mw) of the blend may be at least 400 Daltons (Da), preferably at least 500 Da, more preferably at least 600 Da, particularly at least 700 Da. The weight average molecular weight of the blend may be at most 100,000 Da, preferably at most 50,000 Da, more preferably at most 10,000 Da, particularly at most 5,000 Da, especially at most 2,500 Da, yet further preferably at most 1500 Da, even further preferably at most 1200 Da. The molecular weight may be measured by size exclusion chromatography such as size-exclusion HPLC (SE-HPLC) as described in the Test Methods below.

The composition of the invention may comprise at least 2 wt %, preferably at least 4 wt %, more preferably at least 8 wt %, particularly at least 10 wt % of the first component on the basis of the total weight of the composition. The composition may comprise at most 30 wt %, preferably at most 20 wt %, more preferably at most 18 wt % of the first component on the basis of the total weight of the composition.

The composition may comprise at least 1 wt %, preferably at least 2 wt %, more preferably at least 4 wt %, particularly at least 5 wt %, desirably at least 8 wt % of the second component on the basis of the total weight of the composition. The composition may comprise at most 30 wt %, preferably at most 20 wt %, more preferably at most 18 wt %, particularly at most 15 wt % of the second component on the basis of the total weight of the composition.

The first component and the second component in combination (i.e. the blend) may be at least 10 wt %, preferably at least 14 wt %, more preferably at least 16 wt % of the composition, on the basis of the total weight of the composition. The first component and the second component in combination (i.e. the blend) may be at most 40 wt %, preferably at most 30 wt %, more preferably at most 25 wt % of the composition, on the basis of the total weight of the composition.

The composition may comprise water. The composition may comprise at least 10 wt % water, preferably at least 20 wt % water, more preferably at least 30 wt % water, particularly at least 40 wt % water, desirably at least 50 wt % water, especially at least 60 wt % water, yet further preferably at least 70 wt % water on the basis of the total weight of the composition. The composition may comprise at most 90 wt % water, preferably at most 80 wt % water on the basis of the total weight of the composition.

The composition may further comprise one or more preservatives. The composition may comprise at least 0.1 wt % preservative, preferably at least 0.2 wt %, more preferably at least 0.4 wt %, particularly at least 0.6 wt % on the basis of the total weight of the composition. The composition may comprise at most 10 wt % preservative, preferably at most 5 wt %, more preferably at most 3 wt %, particularly at most 2 wt % on the basis of the total weight of the composition. The preservative may comprise phenoxyethanol, sodium benzoate or potassium sorbate, preferably the preservative comprises sodium benzoate and/or potassium sorbate. Alternatively, the composition may not comprise a preservative.

The composition may be a liquid at room temperature (25° C.). The composition may be a solution or dispersion, preferably a solution, of the blend in a solvent, preferably the solvent comprises water.

Alternatively the composition may be a solid at room temperature (25° C.). The composition may be a powder, preferably a powder obtained by spray drying.

Personal Care and/or Hair Care Formulations

The invention may provide a personal care formulation comprising the composition of the invention and a personal care ingredient. Preferably the personal care formulation is for topical application to skin or hair.

The personal care ingredient may be selected from a cleaning agent, hair conditioning agent, hair styling agent, anti-dandruff agent, hair growth promoter, perfume, sunscreen, sunblock, pigment, moisturizer, film former, hair color, make-up agent, thickening agent, emulsifier, humectant, emollient, antiseptic agent, deodorant active, dermatologically acceptable carrier, surfactant, abrasive, absorbent, fragrance, colorant, essential oil, astringent, anti-acne agent, anti-caking agent, anti-foaming agent, anti-oxidant, binder, enzyme, enzyme inhibitor, enzyme activator, coenzyme, botanical extract, ceramide, buffering agent, bulking agent, chelating agent, cosmetic biocide, external analgesic, substantivity increasing agent, opacifying agent, pH adjuster, reducing agent, sequestrant, skin bleaching and/or lightening agent, skin conditioning agent, skin soothing and/or healing agent, skin treating agent, vitamin or preservative.

Preferably the personal care ingredient is selected from a cleaning agent, hair conditioning agent, skin conditioning agent, hair styling agent, antidandruff agent, hair growth promoter, perfume, sunscreen compound, pigment, moisturizer, film former, humectant, alpha-hydroxy acid, hair colour, make-up agent, thickening agent, antiseptic agent, deodorant, surfactant.

The personal care formulation may be a skin care and/or hair care formulation, preferably a hair care formulation.

The personal care formulations of the type defined herein may be in the form of emulsions (such as oil in water emulsions or water in oil emulsions); anhydrous formulations (such as hair oils, hair sprays/serums); detergent formulations; preferably oil in water emulsions and/or detergent formulations. Personal care emulsion formulations can take the form of pastes, creams, liquids and milks desirably, and in the field of hair care formulations aim to provide a pleasant aesthetic feel to the hair as well as improving manageability and visual appearance.

The personal care formulation may have a range of different consistencies and/or viscosities depending on the desired end use of the formulation. The personal care formulation is preferably a fluid at room temperature. The personal care formulation may be a powder or solid formulation at room temperature.

The end use applications of such formulations include, in the field of personal care products, moisturizers, body butters, gel creams, high perfume containing products, perfume creams, hair conditioners, hair relaxer formulations, hair shampoos, hair styling products, leave-on hair products, water-free products, anti-perspirant and deodorant products, cleansers, 2-in-1 foaming emulsions, emulsifier free products, mild formulations, scrub formulations e.g. containing solid beads, silicone in water formulations, pigment containing products, sprayable emulsions such as hair detanglers, colour cosmetics, shower products, make-up remover, eye make-up remover, and wipes. More preferably, the end use applications of such formulations include hair conditioners, hair relaxer formulations, hair shampoos, hair styling products, leave-on hair products and sprayable emulsions such as hair detanglers. Preferably the personal care formulation is selected from a shampoo, a leave-on conditioner, a rinse-off conditioner, and a hair styling product.

Preferably, the composition of the invention is the only active conditioning ingredient present in the personal care formulation. Preferably, the formulation is free from additional conditioning components, for example quaternised ammonium compounds or silicones.

Preferably, the composition of the invention is present at a low concentration in the personal care (or hair care) formulation. Preferably, the composition of the invention is present in the formulation at a concentration of at least 0.01% w/w, preferably at least 0.1% w/w, more preferably at least 0.5% w/w and most preferably at least 0.8% w/w based on the total weight of the formulation. Preferably, the composition of the invention is present in the formulation at a concentration of up to 40% w/w, up to 30% w/w, up to 20% w/w, preferably up to 15% w/w, more preferably up to 10% w/w and most preferably up to 8% w/w based on the total weight of the formulation.

Preferably, the personal care (or hair care) formulation comprises a base vehicle to carry the composition of the invention. Preferably, the base vehicle comprises a relatively high concentration of water. Preferably, water is present in the personal care formulation at a concentration of at least 20% w/w, preferably at least 25% w/w, more preferably at least 28% w/w and most preferably at least 30% w/w of the total formulation. Preferably, water is present in the personal care formulation at a concentration of up to 99% w/w, preferably up to 96% w/w, preferably, up to 94% w/w and most preferably up to 90% w/w of the total formulation. Alternatively, the formulation may be substantially water free. The formulation may not comprise water.

Preferably, the personal care (or hair care) formulation is acidic. Preferably the formulation has a pH of between 1 and 6, preferably between 2 and 5.5, more preferably of between 3 and 5, and most preferably of between 4 and 4.8.

The personal care (or hair care) formulation may comprise additional components, for example, one or more emulsifiers, emollients, carriers, surfactants and the like.

Preferably, the personal care (or hair care) formulation further comprises an emulsifier. Preferably, the emulsifier is a non-ionic, high HLB (hydrophilic/lipophilic balance) surfactant which is capable of forming an oil-in-water emulsion. The emulsifier may be naturally derived. Examples of suitable emulsifiers include ethoxylated sorbitan esters, ethoxylated glyceryl esters, ethoxylated fatty alcohols (including lanolin alcohols), ethoxylated fatty acids (including lanolin fatty acids), glycerol fatty acid mono-esters, glycol fatty acid mono and di-esters, sugar esters (fatty acid mono and di esters of sucrose), fatty acid polyol (polyethylene glycol) esters, fatty alcohols (which may also act as co-emulsifiers), fatty acids and/or phosphate esters thereof, cationic surfactants or monoalkyl tertiary amines such as stearamidopropyl dimethylamine or behenamidopropyl dimethylamine.

When present in the formulation, the emulsifier is preferably present at a concentration of at least 0.2% w/w, preferably at least 0.5% w/w, more preferably at least 0.9% w/w and most preferably at least 1.1% w/w based on the total weight of the formulation. Preferably, the emulsifier is present in the formulation at a concentration of up to 20% w/w, preferably up to 12% w/w, more preferably up to 7% w/w and most preferably up to 5% w/w based on the total weight of the formulation. The concentration of emulsifier present in the formulation is preferably higher than that present in a formulation of this type comprising quaternised materials. This is to compensate for the absence of quaternised materials in the formulation which would usually have an emulsifying effect on the formulation.

The personal care (or hair care) formulation may further comprise at least one co-emulsifier. Preferably, the or each co-emulsifier is a viscosity modifier, able to modify the viscosity of the formulation, more preferably a viscosity builder, able to increase the viscosity of the formulation. Preferably, the or each co-emulsifier is a fatty alcohol, preferably a C₁₂ to C₂₀ alcohol, more preferably a C₁₆ to C₁₈ alcohol, or a mixture thereof. Suitable alcohols for use as co-emulsifiers in the personal care formulation include cetyl alcohol, stearyl alcohol and cetearyl alcohol.

The formulations according to the present invention may also contain other additional emollient materials, preferably emollient oils. Preferably, the emollient oil is a non-polar oil. Examples of emollient oils which are suitable for use in the present formulation include mineral or paraffin oil; esters of fatty acids and fatty alcohols, preferably C₁₀-C₂₀ acids or alcohols, although isopropyl esters may be used; fatty acid glycol esters; fatty acid triglycerides; esters and diesters of alkoxylated fatty alcohols; botanical (plant) extracts; and hydrocarbons, preferably C₁₂-C₁₆. Preferably, the emollient is mineral oil. When present in the formulation, the or each additional emollient is preferably present at a concentration of at least 1% and up to 30% by weight based on the total weight of the formulation.

The personal care (or hair care) formulation according to the present invention may also contain one or more surfactants, for example sodium lauryl ether (or laureth) sulphate and/or cocamidopropyl betaine. When present in the formulation, the or each surfactant is preferably present at a concentration of between 1% and 20%, preferably between 2% and 15% and more preferably between 4% and 10% by weight based on the total weight of the formulation.

The personal care (or hair care) formulation according to the present invention may also contain one or more cationic ingredients. When present in the formulation, the or each cationic ingredient is preferably present at a concentration of between 0.01% and 10%, preferably between 0.05% and 8% and more preferably between 0.1% and 5% by weight based on the total weight of the formulation.

The personal care (or hair care) formulation according to the present invention may also contain one or more silicones. When present in the formulation, the or each silicone is preferably present at a concentration of between 0.05% and 10%, preferably between 0.1% and 8% and more preferably between 0.5% and 5% by weight based on the total weight of the formulation.

The personal care (or hair care) formulation according to the present invention may also contain one or more film forming components. When present in the formulation, the or each film forming component is preferably present at a concentration of between 0.01% and 5%, preferably between 0.05% and 3% and more preferably between 0.1% and 2% by weight based on the total weight of the formulation.

Many other components that may be used in the formulations according to the present invention. These components may be oil soluble, water soluble or non-soluble. Examples of such materials include:
(i) preservatives, preferably those approved for cosmetic use, particularly as listed in Annex 5 of the European Union cosmetics regulations. The preservative preferably comprises sodium benzoate, potassium sorbate or phenoxyethanol. The preservative may comprise parabens (alkyl esters of 4-hydroxybenzoic acid), substituted ureas or hydantoin derivatives, e.g. those sold commercially under the trade names Germaben II, Nipaguard BPX and Nipaguard DMDMH. The preservative may be used at a concentration in the range from 0.5 wt. % to 2 wt. % based on the total weight of the composition;
(ii) perfumes, when used typically at a concentration in the range from 0.1 wt. % to 10 wt. % more usually up to about 5 wt. % and particularly up to about 2 wt. %, based on the total weight of the composition;
(iii) humectants or solvents such as alcohols, polyols such as glycerol and polyethylene glycols, when used typically at a concentration in the range from 1 wt. % to 10 wt. % based on the total weight of the composition;
(iv) alpha hydroxy acids such as glycolic, citric, lactic, malic, tartaric acids and their esters; self-tanning agents such as dihydroxyacetone;
(v) vitamins and their precursors including: (a) Vitamin A, e.g. as retinyl palmitate and other tretinoin precursor molecules, (b) Vitamin B, e.g. as panthenol and its derivatives, (c) Vitamin C, e.g. as ascorbic acid and its derivatives, (d) Vitamin E, e.g. as tocopheryl acetate, (e) Vitamin F, e.g. as polyunsaturated fatty acid esters such as gamma-linolenic acid esters;
(vi) skin care agents such as ceramides either as natural materials or functional mimics of natural ceramides;
(vii) natural phospholipids, e.g. lecithin;
(viii) vesicle-containing formulations;
(ix) botanical extracts with beneficial skin care properties;
(x) skin whiteners such as kojic acid, arbutin and similar materials;
(xi) skin repair compounds actives such as Allantoin and similar series;
(xii) caffeine and similar compounds;
(xiii) cooling additives such as menthol or camphor;
(xiv) insect repellents such as N,N-diethyl-3-methylbenzamide (DEET) and citrus or eucalyptus oils;
(xv) essential oils; and
(xvi) pigments, including microfine pigments, particularly oxides and silicates, e.g. iron oxide, particularly coated iron oxides, and/or titanium dioxide, and ceramic materials such as boron nitride, or other solid components, such as are used in make-up and cosmetics, to give suspoemulsions, typically used in an amount in the range from 1 wt. % to 15 wt. %, but usually at least 5 wt. %, and particularly about 10 wt. % based on the total weight of the formulation.

The formulations may comprise a fragrance-imparting material to provide a pleasant scent. In one aspect, a scent is provided from a natural source, such as but not limited to alfalfa, almond, amber, angelica root, anise, apple, apricot, banana, basil, bay, bay laurel, benzoin, bergamot, bitter orange, black pepper, bois de rose (rosewood), cajeput, cardamom, carrot seed, cedarwood, cinnamon, citronella, citrus, clary sage, clove, cocoa, coconut, coffee, coriander, cranberry, cypress, elemi, eucalyptus globulous, eucalyptus, fennel, frankincense, galbanum, geranium, German chamomile, ginger, grapefruit, helichrysum, hyssop, jasmine, juniper berry, lavender, lemon, lemongrass, lily, linden blossom, mango, marjoram, melissa, mint, myrrh, myrtle, neroli, niaouli, nutmeg, orange, oregano, palm, parsley, patchouli, peach, peppermint, petitgrain, pine, pineapple, raspberry, Roman chamomile, rose, rosemary, sandalwood, spearmint, spruce, strawberry, tea, thyme, vanilla, vetiver, violet, yarrow, ylang ylang, and the like. Preferably, the fragrance is selected from mint or vanilla.

The invention may provide a hair care formulation comprising the composition of the invention and a hair care ingredient. Preferably the hair care formulation is for topical application to hair.

The hair care ingredient may be selected from shine enhancers, moisturisers, herbal additives, hair strengtheners, vitamin additives, colorants, hair thickening agents; setting and styling agents; ultraviolet absorbers; silicone oils; essential oils and fragrances; thickening or viscosity-enhancing agents; detergents; stabilising agents; emollients; chelating agents; sequestering agents; preservatives; disinfectants; anti-oxidants/radical scavengers; antistatic agents; conditioning agents; detangling ingredients; emulsifying or dispersing agents.

Preferably the hair care formulation is a hair cleansing, conditioning, detangling, colour-protecting or styling formulation, particularly preferably a hair cleansing or conditioning formulation. Preferably the hair care formulation is selected from a shampoo, a leave-on conditioner, a rinse-off conditioner, and a hair styling product.

Preferably, the composition of the invention is present at a low concentration in the hair care formulation. Preferably, the composition of the invention is present in the formulation at a concentration of at least 0.01% w/w, preferably at least 0.1% w/w, more preferably at least 0.5% w/w and most preferably at least 0.8% w/w based on the total weight of the formulation. Preferably, the composition of the invention is present in the formulation at a concentration of up to 20% w/w, preferably up to 15% w/w, more preferably up to 10% w/w and most preferably up to 8% w/w based on the total weight of the formulation.

The hair care formulation may comprise a silicone fluid or oil such as dimethylpolysiloxane, dimethyl silicone, highly polymerised methyl polysiloxane, and methyl polysiloxane, known generically as dimethicone, cyclic oligomeric dialkylsiloxanes, such as the cyclic oligomers of dimethylsiloxane, known generically as cyclomethicone. The concentration of silicone oil in the formulation may preferably be in the range from 0.1 wt. % to 40 wt. %, more preferably 0.3 wt. % to 20 wt. %, particularly 0.5 wt. % to 5 wt. %, and especially 1 wt. % to 1.5 wt. % based on the total weight of the formulation. Alternatively, the hair care formulation may not comprise a silicone compound.

The formulation may be in the form of an aqueous “leave on” or an aqueous “rinse off” end-use product. For such formulations, a dilute solution may be used. Preferably, a buffered solution is used, in which the pH of the solution is adjusted to mildly acidic, with a pH in the range of from 4 to 6. In the case of rinse-off formulations, instructions are provided to wash off the diluted formulation after application. Depending on the level of treatment required, such instructions may also require the product to remain on the hair for some time, such as from 1 to 30 minutes. For leave-on formulations, the washing off step is omitted.

Where the formulation is a hair shampoo or conditioner which functions to make the hair straighter, the shampoo or conditioner may be in the form of a dispersion, emulsion or solution. One preferred system is one that forms liquid crystals. The liquid crystals are preferably lyotropic liquid crystals (i.e. both concentration and temperature dependent), more preferably lamellar phase liquid crystals, and particularly L alpha phase (neat) liquid crystals.

The formulation may contain many different types of functional ingredients such as;

(i) cationic hair conditioning agents, e.g. ethoxylated phosphate fatty quats, such as those sold by Croda as Arlasilk™; fatty amido amines, such as those sold by Croda as Incromine™; fatty quats, such as those sold by Croda as Incroquat™, Crodazosoft™, Rejuvasoft™ or VibraRiche™ typically used at a concentration in the range from 1 wt. % to 5 wt. % based on the total weight of the composition. These are typically combined with polymeric hair conditioning cationic materials such as quaternised cellulose sold by Croda as Crodacel™ and MiruStyle™, quaternised proteins, such as those sold by Croda, as Croquat™, Crosilkquat™, Kerestore™ and Hydrotriticum™.

(ii) fatty alcohols, e.g. stearyl, cetearyl, cetyl, oleyl alcohols, used typically at a concentration range of 2 wt. % to 5 wt. % based on the total weight of the composition.

(iii) humectants or solvents, e.g. alcohols and polyols such as glycerol and polyethylene glycols, when used typically at a concentration in the range from 1 wt. % to 10 wt. % based on the total weight of the composition;

(iv) reconstructors, e.g. hydrolysed proteins such as wheat protein, which function to penetrate the hair and strengthen the hair structure through polymer crosslinking;

(v) glossing or detangling materials which bind to the hair and reflect light, e.g. silicones such as dimethicone, phenyltrimethicone, dimethiconol and/or trimethylsilylamodimethicone, usually at a concentration in the range from 0.2 wt. % to 10 wt. % based on the total weight of the composition;

(vi) acidity regulators, e.g. citric acid, lactic acid, which generally maintain the pH of the conditioner at about 4 to 6;

(vii) thermal protectors, usually heat-absorbing polymers, which shield the hair against excessive heat, e.g. caused by blow-drying or curling irons or hot rollers such as for instance those sold by Croda as Mirustyle™ MFP (quaternised starch); and

(viii) UV protection agents, to protect hair or formulation components from degradation by UV light, such as those sold by Croda as Crodasorb™ UV-HPP.

Method of the Invention

According to a second aspect, the invention provides a method of treating hair comprising the steps of:
i) applying to hair a composition comprising a first partially hydrolysed protein component obtained from a potato protein source and a second partially hydrolysed protein component obtained from a non-potato vegetable protein source; and
ii) optionally, rinsing the composition off the hair.

The method of treating hair may comprise one or more steps selected from bleaching, colouring, straightening, perming, curling, relaxing, fixing, holding, shaping, styling, thermal treatment, cleaning or conditioning of hair. Preferably the method of treating hair comprises bleaching, colouring, shaping, styling, cleaning or conditioning of hair, more preferably shaping, styling, cleaning or conditioning of hair, particularly preferably cleaning or conditioning of hair.

The hair may be natural, bleached, straightened or otherwise chemically treated. The hair may be African, Asian, European, Caucasian, Hispanic or another hair type. The hair may be naturally straight, wavy, curly or coily.

Preferably, the application of the composition to the hair strengthens the hair, more preferably the application of the composition to the hair increases the average total work to break of the hair, particularly measured as described herein.

The composition may comprise any of the features described herein.

Use of the Invention

According to a third aspect, the invention provides the use of a composition comprising a first hydrolysed protein component derived from potato and a second hydrolysed protein component derived from a non-potato vegetable source for the treatment of hair.

The treatment of hair may comprise strengthening the hair. Preferably the treatment of hair comprises increasing the average cross-sectional area of a hair fiber and/or increasing the average total work to break of the hair, particularly measured as described herein. Preferably the treatment of hair comprises increasing the total work to break of the hair, particularly as measured herein.

The composition may comprise any of the features described herein.

Any of the features described herein may be taken in any combination and with any aspect of the invention.

### EXAMPLES

The invention is illustrated by the following non-limiting examples. All parts and percentages are given by weight unless otherwise stated.

It will be understood that all tests and physical properties listed have been determined at atmospheric pressure and ambient/room temperature (i.e. about 23-25° C.), unless otherwise stated herein, or unless otherwise stated in the referenced test methods and procedures.

Test Methods

In this specification the following test methods have been used:
(i) Viscosity was measured at room temperature (25° C.) with Brookfield Ametek DV-1 Viscometer using an appropriate spindle (T-C bar). The sample was tested at 10 rpm (0.17 Hz) for 1 minute, immediately and 24 hours after making the sample and results are quoted in cP (mPa·s).
(ii) pH was measured at room temperature (25° C.) with Fisher Scientific Accumet AE150 pH Meter. Depending on the initial pH reading, the pH of the formulation was adjusted to the range of 4.0-5.0 using 10 wt % sodium hydroxide solution (if the pH is higher) or 10 wt % citric acid solution (if the pH is lower). The pH was measured immediately and 24 hours after making the formulation.
(iii) Actives content (in wt %) in the sample was calculated from measuring the total non-volatile content & ash content. Total non-volatile content was measured for a known weight of sample by oven drying at 105° C. for 17-19 hours to remove the moisture and any other volatile components present. After cooling in a desiccator, the residual weight is used to calculate the total non-volatile content (in wt %) of the sample. For ash content, a known weight of sample is heated carefully, using incremental temperature steps, to a temperature of 575-600° C. in the presence of air and held at temperature for 16-18 hours (or longer if necessary). After cooling in a desiccator, the residual weight is used to calculate the ash content (in wt %) of the sample. The actives content (in wt %) in the sample is then calculated by subtracting wt % ash from wt % total non-volatile content.
(iv) Weight average molecular weight was determined by Size-Exclusion High-Performance Liquid Chromatography (SE-HPLC). The HPLC apparatus and settings used are given in Table 1 below.
(v) Amino acid profile was measured by fully hydrolysing the sample into constituent amino acids. The distribution of these amino acids was then quantified via HPLC.
(vi) Single hair fiber tensile test data was measured using a Miniature Tensile Tester (MTT686), equipped with Automatic Loading System (model ALS1500) and Fiber Dimensional Analysis System (model FDAS770) from Diastron Ltd. (Andover, Hants, UK) in a controlled environment chamber. 50 random single fibers from each hair tress treated with each sample were first prepared for testing with AAS 1600 (Auto-Assembly System) from Diastron Ltd. Each hair fiber sample had specification of 30 mm in length. The prepared hair fiber samples were then loaded onto the rotary sample tray and pre-equilibrated at 50% relative humidity (RH) and room temperature (25° C.) for 2 hours prior to performing the experiment. After pre-equilibration for 2 hours, each fiber sample is moved from the sample tray to FDAS770 on automated fashion using ALS1500, and as FDAS770 employs a laser micrometer, dimensions of the fiber sample (cross-sectional area in square microns) were measured by scanning 3 slices along the length of the fiber. The fiber sample was then moved back to the sample tray, where a pneumatically operated rod holds one end of the fiber sample taut while a pneumatically operated jaw equipped with 2 kg load cell grabs onto the other end of the fiber sample, and slowly stretched the hair fiber sample up to 2% extension at 20 mm/min extension rate. After each fiber sample has undergone 2% extension, the same fiber sample was stretched until the fiber reached break point. At break point, each fiber sample generated stress-strain curve. Data for Total Work to Break (measured in unit of Joules) was generated, which is defined as the integral area under the stress-strain curve or maximum work required to break the hair fiber sample.

TABLE 1
HPLC apparatus and settings

| | |
|---|---|
| Spectrometer | Agilent 1200 series HPLC |
| Detector | UV @ 220 nm |
| Columns | Superdex 30 Increase |
| Solvent | 0.1% TFA, 0.1M Na₂SO₄, 22% MeCN |
| Concentration of test | 0.02 g/ml |
| substance | |
| Column temperature | 25° C. |
| Flow rate | 0.8 ml/min |
| Injection Volume | 10 μL |
| Analysis time | 45 minutes |
| Method control and | Agilent OpenLab software |
| integration | |
| Method Type | Peak area integration |
| Calibration | Narrow standard calibration, 3^{rd} order |
| | polynomial, using protein standards |
| | cytochrome C, insulin chain B, bacitracin, |
| | vitamin B12, Arg-Gly-Asp-Ser, Gly-Gly-Gly, |
| | glutamine, glycine |

### Example 1: Preparation of Samples B to G According to the Invention

Samples B to G according to the invention were prepared as follows and their compositions are given in Table 2 below.

Sample B

Potato protein concentrate was added to water and mixed until well dispersed. The resulting dispersion was heated to the desired temperature before the pH was adjusted using sodium hydroxide solution. Enzymes (carbohydrase and protease) were used to catalyse hydrolysis of the potato protein by stirring at the desired temperature and pH ranges to achieve a typical weight average molecular weight of about 600 to 800 Da. Following hydrolysis, the pH of the hydrolysis mixture was lowered to acidic through addition of hydrochloric acid solution and the enzymes were denatured via heating. The protein hydrolysate was purified by filtration and treatment with activated carbon. The resulting dilute hydrolysed protein solution was then concentrated to the desired actives content (in the range 15 to 25 wt % actives content) and preserved.

Wheat protein concentrate was added to water and mixed until well dispersed. The resulting dispersion was heated to the desired temperature before the pH was adjusted using sodium hydroxide solution. A protease enzyme was used to catalyse hydrolysis of the wheat protein by stirring at the desired temperature and pH ranges. Following hydrolysis, the pH of the hydrolysis mixture was lowered to acidic through addition of hydrochloric acid solution and the enzyme was denatured via heating. The protein hydrolysate was purified by filtration, treatment with activated carbon and ion-exchange. The resulting dilute hydrolysed protein solution was then concentrated to the desired actives content (in the range 15 to 25 wt % actives content) and preserved.

The hydrolysed potato protein solution and hydrolysed wheat protein solution were blended together in a 2:1 weight ratio by actives content to give Sample B.

Sample C

Sample C was prepared in the same way as Sample B but using a 1:1 weight ratio by actives content.

Sample D

Hydrolysed potato protein was prepared as per Sample B.

Pea protein concentrate was added to water and mixed until well dispersed. The resulting dispersion was heated to the desired temperature before the pH was adjusted using sodium hydroxide solution. A protease enzyme was used to catalyse hydrolysis of the pea protein by stirring at the desired temperature and pH ranges to achieve a typical weight average molecular weight of about 800 to 1500 Da. Following hydrolysis, the pH of the hydrolysis mixture was lowered to acidic through addition of hydrochloric acid solution and the enzyme was denatured via heating. The protein hydrolysate was purified by filtration and treatment with activated carbon. The resulting dilute hydrolysed protein solution was then concentrated to the desired actives content (in the range 15 to 25 wt % actives content) and preserved.

The hydrolysed potato protein and hydrolysed pea protein were blended together in a 1:1 weight ratio by actives content to give Sample D.

Sample E

Hydrolysed potato protein was prepared as per Sample B.

Cottonseed protein concentrate was added to water and mixed until well dispersed. The resulting dispersion was heated to the desired temperature before the pH was adjusted using sodium hydroxide solution. Protease enzymes were used to catalyse hydrolysis of the cottonseed protein by stirring at the desired temperature and pH ranges for a defined time period. Following hydrolysis, the pH of the hydrolysis mixture was lowered to acidic through addition of hydrochloric acid solution and the enzymes were denatured via heating. The protein hydrolysate was purified by filtration. The resulting dilute hydrolysed protein solution was then concentrated to the desired actives content (in the range 15 to 25 wt % actives content) and preserved.

The hydrolysed potato protein and hydrolysed cottonseed protein were blended together in a 1:1 weight ratio by actives content to give Sample E.

Sample F

Sample F was prepared in the same way as Sample D but using a 2:1 weight ratio by actives content.

Sample G

Sample G was prepared in the same way as Sample D but using a 1:2 weight ratio by actives content.

TABLE 2
Composition of Samples B to G

| | | Total actives |
|---|---|---|
| | Composition (weight ratios | content in |
| Sample | by actives content) | sample (wt %) |
| B | 2:1 blend of hydrolysed potato | 21.4 |
| | protein and hydrolysed wheat | |
| | protein | |
| C | 1:1 blend of hydrolysed potato | 21.5 |
| | protein and hydrolysed wheat | |
| | protein | |
| D | 1:1 blend of hydrolysed potato | 19.8 |
| | protein and hydrolysed pea | |
| | protein | |
| E | 2:1 blend of hydrolysed potato | 20.2 |
| | protein and hydrolysed | |
| | cottonseed protein | |
| F | 2:1 blend of hydrolysed potato | 20.6 |
| | protein and hydrolysed pea | |
| | protein | |
| G | 1:2 blend of hydrolysed potato | 20.6 |
| | protein and hydrolysed pea | |
| | protein | |

### Example 2: Comparative Products

Comparative products were obtained as shown in Table 3 below.

TABLE 3
Composition of comparative products

| Comparative | |
|---|---|
| Product | Description |
| 1 | Hydrolysed Vegetable Protein |
| 2 | Enzyme Hydrolysed Keratin |

### Example 3: Amino Acid Profiles

The samples B to G of the invention and comparative product 2 were fully hydrolysed into their constituent amino acids and the distribution of these amino acids (% by weight on the basis of the total weight of amino acids in the sample) were then quantified via HPLC analysis. These amino acid profiles are given in Table 4 below.

TABLE 4
Amino acid profiles

| | Sample/Comparative Product | | | | | | |
|---|---|---|---|---|---|---|---|
| | B | C | D | E | F | G | 2 |
| Aspartic acid | 9.1 | 7.6 | 12.2 | 11.5 | 12.2 | 12.3 | 7.7 |
| Threonine | 4.9 | 4.4 | 5.1 | 5.4 | 5.4 | 4.8 | 6.1 |
| Serine | 5.9 | 5.8 | 6.1 | 5.9 | 6.1 | 6.2 | 9.5 |
| Glutamic acid | 21.4 | 25.3 | 16.2 | 16.6 | 15.3 | 17.1 | 15.0 |
| Proline | 7.6 | 8.7 | 4.9 | 4.8 | 5.0 | 4.7 | 6.1 |
| Glycine | 3.9 | 3.7 | 4.1 | 4.3 | 4.2 | 4.0 | 4.3 |
| Alanine | 4.2 | 3.8 | 4.9 | 4.7 | 4.9 | 4.8 | 3.9 |
| Cystine* | 1.1 | 1.4 | 1.2 | 0.8 | 1.0 | 1.4 | 11.0 |
| Valine | 4.9 | 4.7 | 5.2 | 5.2 | 5.3 | 5.0 | 5.2 |
| Methionine | 2.1 | 2.1 | 1.6 | 1.9 | 1.8 | 1.4 | 0.0 |
| Isoleucine | 4.0 | 3.9 | 4.2 | 3.9 | 4.3 | 4.2 | 3.3 |
| Leucine | 8.6 | 8.2 | 8.8 | 8.4 | 9.0 | 8.5 | 8.0 |
| Tyrosine | 4.4 | 4.2 | 3.7 | 4.2 | 4.1 | 3.2 | 3.6 |
| Phenylalanine | 5.1 | 5.3 | 4.2 | 5.0 | 4.4 | 4.0 | 3.1 |
| Histidine | 2.4 | 2.2 | 2.8 | 3.5 | 2.8 | 2.8 | 1.3 |
| Lysine | 5.7 | 4.7 | 8.1 | 6.5 | 8.0 | 8.2 | 3.0 |
| Arginine | 4.6 | 4.3 | 6.8 | 7.5 | 6.3 | 7.3 | 8.9 |

*cystine can also include cysteine & cysteic acid

It can be seen that the amounts of cystine in vegetable derived Samples B to G are lower than in comparative product 2 which is animal derived hydrolysed keratin. However, as can be seen in Example 4, the strength of hair (as demonstrated by improved total work to break) was increased by Samples B to G when compared with comparative product 2.

### Example 4: Hair Tensile Test

Hair samples were treated and tensile tested using the following procedure:

a) Saturate each hair tress (1.0 cm wide Regular Bleached Caucasian Hair from same lot) in a 1 wt % actives content diluted solution of each sample for 1 hour

b) the untreated control hair tress was also saturated in deionised (DI) water for 1 hour

c) after 1 hour, each tress was rinsed off under lukewarm running water at constant flow rate for 30 seconds

d) Each treated hair tress was air dried overnight inside controlled humidity environment set at 50% RH.

Hair tensile test data was measured as described in the Test Method section above. Results were obtained for fifty (50) random single fibers from each pre-bleached human hair tress. Each fiber was treated with a 1 wt % actives content aqueous solution of comparative products 1 & 2 and samples B to G of the invention. Untreated bleached hair fibers were also tested. The average results are given in Table 5 below.

TABLE 5
Hair Tensile Test Results

| Comparative | | |
|---|---|---|
| product/ | Average Cross- | Average Total |
| Sample of the | Sectional Area | Work to Break |
| Invention | (sq. microns) | (J) |
| Untreated | 3668 | 7.64 × 10⁻³ |
| Bleached Hair | | |
| 1 | 3899 | 8.26 × 10⁻³ |
| 2 | 3653 | 7.99 × 10⁻³ |
| B | 4353 | 9.05 × 10⁻³ |
| C | 4667 | 9.58 × 10⁻³ |
| D | 4408 | 9.63 × 10⁻³ |
| E | 4226 | 8.55 × 10⁻³ |
| F | 4082 | 8.66 × 10⁻³ |
| G | 4289 | 9.30 × 10⁻³ |

It can be seen from Table 5 that the average cross-sectional area of the hair fibers treated with samples B to G is increased when compared with the untreated fibers or comparative products 1 & 2. This indicates increased substantivity of the hair. Furthermore, the work required to break hair fibers treated with samples B to G is also increased which indicates that the hair has been strengthened.

### Example 5: Hair Conditioner Formulation

A basic hair conditioner formulation was produced as shown in Table 6 below.

TABLE 6
Basic Hair Conditioner Formulation

| Ingredient | Amount (wt %) |
|---|---|
| Deionized Water | Q.S. to 100% |
| Cetrimonium Chloride | 5.00% |
| Cetosteary Alcohol | 4.50% |
| Sample of the Invention | Equivalent to 1 wt % actives |
| | content or 0% for Placebo |
| | Conditioner |
| Phenoxyethanol and | 1.00% |
| Ethylhexylglycerin | |
| (Preservative) | |

The following basic hair conditioner formulations given in Table 7 below were made and tested for viscosity and pH as described in the Test Methods above.

TABLE 7
Hair Conditioner Formulations

| | | | | Final |
|---|---|---|---|---|
| | | Final | Initial | Viscosity |
| | | pH after | Viscosity | after 24 h |
| | Initial pH | 24 h | (cps) | (cps) |
| Placebo | 4.3 | 4.36 | 15,900 | 31,800 |
| Conditioner | | | | |
| Conditioner | 4.29 | 4.33 | 15,200 | 26,700 |
| with Sample | | | | |
| C | | | | |
| Conditioner | 4.45 | 4.54 | 15,700 | 32,300 |
| with Sample | | | | |
| D | | | | |

These basic hair conditioner formulations were tested using the following procedure:

a) Apply each conditioner at controlled dosage of 2.5:10 w/w conditioner to hair ratio per standardized PVCS protocol PVCS-002.

b) Each conditioner was massaged onto the hair for 1 minute.

c) The conditioner was left on the hair for 20 minutes prior to rinse off.

d) After 20 minutes, each tress was rinsed off under lukewarm running water at constant flow rate for 30 seconds.

e) Each treated hair tress was air dried overnight inside controlled humidity environment set at 50% RH.

Tensile testing was then performed as described in the Test Method section above. The results are given in Table 8 below.

TABLE 8
Conditioner Hair Tensile Test Results

| | Average Cross- | Average Total Work |
|---|---|---|
| | Sectional Area | to Break |
| Treatment | (sq. microns) | (J) |
| Untreated Bleached Hair | 3984.55 | 8.84 × 10⁻³ |
| Placebo Conditioner | 4020.36 | 8.71 × 10⁻³ |
| Conditioner with Sample C | 4469.96 | 9.99 × 10⁻³ |
| Conditioner with Sample D | 4278.11 | 9.26 × 10⁻³ |

It can be seen from Table 8 that the average cross-sectional area of the hair fibers treated with the conditioner using samples C & D is increased when compared with the placebo conditioner. This indicates increased substantivity of the hair. Furthermore, the work required to break hair fibers treated with the conditioner using samples C & D is also increased which indicates that the hair has been strengthened.

### Example 6: Personal Care Formulations

Sample D was included in various personal care formulations as shown in Tables 9 to 11 below, which all passed a stability test of 1 month at 50° C. and 5×24 hour freeze-thaw cycles.

TABLE 9
Shampoo

| Ingredient/INCI Name | Functionality | % w/w |
|---|---|---|
| Part A | | |
| Water Deionised (Aqua) | — | To 100 |
| Crodasinic LS30 (Aqua (and) Sodium Lauroyl | Surfactant | 20.00 |
| Sarcosinate)¹ | | |
| Crodateric CAB 30 (Cocamidopropyl Betaine | Surfactant | 20.00 |
| (and) Water (aqua))¹ | | |
| Crodateric CAS 50 (Cocamidopropyl | Surfactant | 10.00 |
| Hydroxysultaine)¹ | | |
| Versathix (PEG-150 Pentaerythrityl Tetrastearate | Thickener | 4.00 |
| (and) PPG-2 Hydroxyethyl Cocamide (and) Aqua)¹ | | |
| Euxyl ® K903 (Benzyl Alcohol (and) Benzoic Acid | Preservative | 1.20 |
| (and) Dehydroacetic Acid (and) Tocopherol)² | | |
| Cromollient ™ SCE (Di-PPG-2 Myreth-10 | Emollient | 1.00 |
| Adipate)¹ | | |
| Sample D | Protein | 1.00 |

Suppliers: 1: Croda 2: Schülke Inc.

Preparation Procedure for Shampoo:

Add water to main beaker. Add remaining ingredients one at a time and allow to mix in completely. Adjust pH if necessary.

TABLE 10
Conditioner

| Ingredient/INCI Name | Functionality | % w/w |
|---|---|---|
| Part A | | |
| Water Deionised (Aqua) | — | To 100 |
| SP Rejuvasoft MBAL (Quaternium-91 (and) | Conditioning | 6.00 |
| Behentrimonium Chloride (and) Myristyl | agent | |
| Myristate (and) Cetearyl Alcohol)¹ | | |
| Part B | | |
| Sample D¹ | Protein | 1.00 |
| Euxyl ® K712 (Aqua (and) Sodium Benzoate | Preservative | 1.00 |
| (and) Potassium Sorbate)² | | |

Suppliers: 1: Croda 2: Schülke, Inc

Preparation Procedure for Conditioner:

Add water to the main beaker. Begin mixing using a propeller blade and heat to 80° C. Once the water has reached 80° C., add SP Rejuvasoft MBAL and allow to mix for 10-15 minutes at high speed. Begin cooling to 60° C. and hold at this temperature for an additional 15 minutes. The emulsion should look smooth with no particles. Once the emulsion looks smooth, continue to cool to room temperature. Once under 40° C., add Part B ingredients one at a time, allowing to fully mix in. Adjust pH using 25% citric acid solution if necessary.

TABLE 11
Hair Mask

| Ingredient/INCI Name | Functionality | % w/w |
|---|---|---|
| Part A | | |
| Water Deionised (Aqua) | — | To 100 |
| Glycerin | Humectant | 1.00 |
| Lactic Acid (85%) | Neutralising agent | 0.20 |
| Part B | | |
| Crodacol ™ CS50 (Cetostearyl Alcohol)¹ | Thickener | 5.00 |
| Crodamine SC (Hydroxyethyl | Conditioning agent | 1.50 |
| Diethylenetriamine | | |
| Dioleamide/Palmitamide)¹ | | |
| SensaLuxe DF (Dioctyldodecyl | Dry friction active | 1.00 |
| Dodecanedioate)¹ | | |
| Crodabond CSA (Hydrogenated Castor | Colour | 1.00 |
| Oil/Sebacic Acid Copolymer)¹ | protection/cuticle | |
| | bonding active | |
| Part C | | |
| Sample D¹ | Protein | 1.00 |
| Euxyl ® PE9010 (Phenoxyethanol (and) | Preservative | 1.00 |
| Ethylhexylglycerin)³ | | |
| Phytessence ™ Hazel Leaf (Glycerin | Scalp revitalizing | 0.50 |
| (and) Water (and) Avellana Leaf Extract)² | botanical | |

Suppliers: 1: Croda 2: Crodarom 3:Schülke, Inc

Preparation Procedure for Hair Mask:

Add Part A ingredients to the main beaker. Begin mixing with a propeller blade and heat to 80° C. In a separate beaker, add Part B ingredients and begin heating to 80° C. Once both phases are at 80° C., slowly pour Part B into Part A with fast mixing. Allow to mix at temperature for 10 minutes and then begin cooling to 60° C. Once the temperature reaches 60° C., switch to a side sweep blade and lower the mixing speed. Continue to cool to 40° C. Once under 40° C., add Part C ingredients one at a time, allowing to fully mix in. Adjust the pH if necessary.

It is to be understood that the invention is not to be limited to the details of the above embodiments, which are described by way of example only. Many variations are possible.

1. A composition comprising a blend of:
a) a first component which is partially hydrolysed protein obtained from a potato protein source; and
b) a second component which is partially hydrolysed protein obtained from a non-potato vegetable protein source;
wherein the weight ratio of the first component:the second component in the blend is in a range 0.5:1 to 2:1.

2. The composition according to claim 1 wherein the weight ratio of the first component:the second component in the blend is in a range 0.6:1 to 1.4:1.

3. The composition according to claim 1 which does not comprise a protein component obtained from an animal protein source.

4. The composition according to claim 1 which does not comprise a protein component obtained from a wheat protein source.

5. The composition according to claim 1 wherein the second component is obtained from a pea or cottonseed protein source.

6. The composition according to claim 1 wherein the first component and/or the second component is a chemically unmodified hydrolysed protein.

7. The composition according to claim 1 wherein the composition is a topical composition for application to skin or hair.

8. The composition according to claim 1 comprising at least 60 wt % water on the basis of the total weight of the composition.

9. A personal care formulation comprising the composition according to claim 1 and a personal care ingredient, wherein the personal care formulation is for topical application to skin or hair.

10. The personal care formulation according to claim 9 wherein the personal care ingredient is selected from a cleaning agent, hair conditioning agent, hair styling agent, anti-dandruff agent, hair growth promoter, perfume, sunscreen, sunblock, pigment, moisturizer, film former, hair color, make-up agent, thickening agent, emulsifier, humectant, emollient, antiseptic agent, deodorant active, dermatologically acceptable carrier, surfactant, abrasive, absorbent, fragrance, colorant, essential oil, astringent, anti-acne agent, anti-caking agent, anti-foaming agent, anti-oxidant, binder, enzyme, enzyme inhibitor, enzyme activator, coenzyme, botanical extract, ceramide, buffering agent, bulking agent, chelating agent, cosmetic biocide, external analgesic, substantivity increasing agent, opacifying agent, pH adjuster, reducing agent, sequestrant, skin bleaching and/or lightening agent, skin conditioning agent, skin soothing and/or healing agent, skin treating agent, vitamin or preservative.

11. A hair care formulation comprising the composition according to claim 1 and a hair care ingredient, wherein the hair care formulation is for topical application to hair.

12. The hair care formulation according to claim 11 wherein the hair care ingredient is selected from shine enhancers, moisturisers, herbal additives, hair strengtheners, vitamin additives, colorants, hair thickening agents; setting and styling agents; ultraviolet absorbers; silicone oils; essential oils and fragrances; thickening or viscosity-enhancing agents; detergents; stabilising agents; emollients; chelating agents; sequestering agents; preservatives; disinfectants; anti-oxidants/radical scavengers; antistatic agents; conditioning agents; detangling ingredients; emulsifying or dispersing agents.

13. A method of treating hair comprising:
i) applying to hair a composition comprising a first partially hydrolysed protein component obtained from a potato protein source and a second partially hydrolysed protein component obtained from a non-potato vegetable protein source; and
ii) optionally, rinsing the composition off the hair.

14. The method according to claim 13 wherein the method comprises one or more steps selected from bleaching, colouring, straightening, perming, curling, relaxing, fixing, holding, shaping, styling, thermal treatment, cleaning or conditioning of the hair.

15. The method according to claim 13 wherein the application of the composition to the hair strengthens the hair.

16.-17. (canceled)
